(19) United States
(12) Patent Application Publication
(10) Pub. No.: US 2026/0108444 A1
(43) Pub. Date: Apr. 23, 2026

(54) ENCAPSULATION COMPOSITIONS AND METHODS FOR ENCAPSULATING ACTIVE AGENTS

(71) Applicant: DEFY-Tech Labs, Inc., Alpharetta, GA (US)

(72) Inventor: Daniel H. TRAYNOR, Alpharetta, GA (US)

(21) Appl. No.: 19/366,174

(22) Filed: Oct. 22, 2025

Related U.S. Application Data
(60) Provisional application No. 63/710,315, filed on Oct. 22, 2024.
(60) Provisional application No. 63/800,873, filed on May 6, 2025.
(60) Provisional application No. 63/883,370, filed on Sep. 17, 2025.
(60) Provisional application No. 63/891,968, filed on Oct. 1, 2025.

(51) Int. Cl. A61K 8/42; A61K 8/11; A61K 8/49; A61K 8/92; A61Q 17/02; A61Q 17/04

(52) U.S. Cl. CPC A61K 8/42; A61K 8/11; A61K 8/4926; A61K 8/922; A61Q 17/02; A61Q 17/04; A61K 2800/48

(57) ABSTRACT

The present disclosure provides compositions and methods useful for delivering and encapsulating active agents. The compositions can comprise a polymer and a medium chain triglyceride (MCT) that comprises fatty acids having 6 to 12 carbon atoms. The compositions can encapsulate active agents including an antibacterial, a color compound, a fragrance compound, an active pharmaceutical ingredient (API), an over-the-counter (OTC) ingredient, an antioxidant, a flavinoid, an essential oil, or a combination thereof. In particular, the compositions can encapsulate essential oils. These compositions provide for the gradual and controlled release of active agents, particularly essential oils and can be used in a variety of topical products including insect repellents.

### CROSS-REFERENCE TO RELATED APPLICATIONS

This application claims priority to U.S. Provisional Patent Application Nos. 63/710,315 filed on Oct. 22, 2024; 63/800,873 filed on May 6, 2025; 63/883,370 filed on Sep. 17, 2025; and 63/891,968 filed on Oct. 1, 2025; the disclosures of each are incorporated herein by reference in their entirety.

### FIELD OF INVENTION

The present disclosure provides compositions and methods useful for delivering and encapsulating active agents. The compositions can comprise a polymer and a medium chain triglyceride (MCT) that comprises fatty acids having 6 to 12 carbon atoms. The compositions can encapsulate active agents including an antibacterial, a color compound, a fragrance compound, an active pharmaceutical ingredient (API), an over-the-counter (OTC) ingredient, an antioxidant, a flavinoid, an essential oil, or a combination thereof. In particular, the compositions can encapsulate essential oils. These compositions provide for the gradual and controlled release of active agents, particularly essential oils and can be used in a variety of topical products including insect repellents.

### BACKGROUND OF INVENTION

Active agents can be a variety of compounds including an antibacterial, a color compound, a fragrance compound, an active pharmaceutical ingredient (API), an over-the-counter (OTC) ingredient, an antioxidant, a flavinoid, or an essential oil. In some instances, these active agents are desired to be delivered at a particular time. In these cases, the active agents can be encapsulated in a coating or partial coating to protect the active agent from evaporation, reaction, oxidation, or otherwise being released prior to the desired time of release.

Essential oils are volatile organic compounds extracted from plant materials and are used in a wide variety of applications. Many have pleasant odors that make them useful for relaxation or aromatherapy. Many also have antibacterial properties, making them useful as preservative agents, and insecticidal properties, making them useful in controlling insect pests.

However, because essential oils have inherent volatility, they have a tendency to release from compositions containing them very quickly. This results in wasted product and a short time frame of effectiveness of each application of the product.

Thus, there exists a need to generate formulations and encapsulation methods that can keep an active agent from being released or degraded during its storage and that allow it to be gradually released upon application of the formulation.

### SUMMARY OF INVENTION

The present disclosure provides a composition comprising: one or more polymers; from about 20 wt. % to about 80 wt. % of one or more medium chain fatty acids or salts or esters thereof having 6-12 carbon atoms; one or more active agents; wherein the active agent and the medium chain fatty acid or salt or ester thereof are coated or partially coated with the polymer.

The present disclosure is further directed to a dry topical composition comprising the composition and at least one carrier. The dry topical composition can be an insect repellent.

Additionally, the present disclosure provides a dry spray dried powder composition comprising: a) molecular sieves impregnated with at least one essential oil; and b) at least one free essential oil; wherein the at least one free essential oil and molecular sieves are coated or partially coated with at least one polysaccharide. The molecular sieves can comprise zeolite particles.

The present disclosure is further directed to a dry topical composition comprising the dry spray dried powder composition and at least one carrier. The dry topical composition can be a sunscreen, an insect repellent, or a combination thereof.

The present disclosure is also directed to a spray-dried composition derived from spray drying an emulsion comprising an oil phase and an aqueous phase, wherein the aqueous phase comprises from about 50 wt. % to about 70 wt. % water and from about 5 wt. % to about 20 wt. % acacia gum, based on the total weight of the emulsion; and the oil phase comprises from about 15 wt. % to about 35 wt. % oil, based on the total weight of the emulsion.

The present disclosure is further directed to methods of use and methods of making these compositions.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 is a Motic camera on a light microscope at 40 times magnification showing spray dried particles prepared from an oil-in-water emulsion that results in a composition having about 50 wt. % acacia gum (i.e., SPRAYGUM™ BA), about 48 wt. % oil (i.e., blend of geraniol, clove bud oil, peppermint oil, thyme oil, cedarwood oil, lemongrass oil, and lauric acid) and about 2 wt. % ALCOLEC® S soy lecithin available from American Lecithin having about 39-45 wt. % phospholipid content.

### DETAILED DESCRIPTION OF INVENTION

This disclosure describes a composition comprising one or more polymers; from about 20 wt. % to about 80 wt. % of one or more medium chain fatty acids or salts or esters thereof having 6-12 carbon atoms; one or more active agents; wherein the active agent and the medium chain fatty acid or salt or ester thereof are coated or partially coated with the polymer.

The composition described herein, wherein the polymer is a polysaccharide polymer.

The composition described herein, wherein the polymer comprises acacia gum, corn starch, carrageenan, hyaluronic acid, pectin, alginate, chitosan, guar gum, arabic gum, xanthan gum, carboxymethylcellulose, carob bean gum, cellulose, alpha-cyclodextrin, polylactic acid, a vinyl polymer, polyethylteraphthalate, or a combination thereof.

The composition described herein further comprising at least two polysaccharide polymers, wherein the polysaccharide polymer comprises acacia gum, corn starch, carrageenan, hyaluronic acid, pectin, alginate, chitosan, guar gum, arabic gum, xanthan gum, carboxymethylcellulose, carob bean gum, cellulose, or alpha-cyclodextrin.

The polysaccharide can be linear or branched with the formula Cₓ(H₂O)_{y}, wherein x is from about 200 to about 2500, wherein y is about 200 to about 2500.

The compositions described herein, wherein the polysaccharide polymer comprises acacia gum.

Arabinogalactan peptide proteins (AGP) are present in acacia gums, also known as glycoproteins, and is an example of a gum that holds the potential for good encapsulation. Other glycoproteins include glycosaminoglycans (GAGs).

Gum Acacia Senegal has a lower % of acacia gum required for good emulsification properties because it has a higher protein content (e.g., 2.5-3.0 wt. % protein) as compared to other acacia gums. Higher protein concentrations of other polymers also provide the same emulsification ability with a lower concentration of the polymer. This emulsification ability is advantageous for retaining the agent (e.g., essential oil or other volatile agent) in the encapsulation composition.

Gum Acacia Seyal of different properties can also be obtained from Nexira, Inc. The Gum Acacia Seyal has approximately 1.0 wt. % protein, and is a more compact molecule as compared to Gum Acacia Senegal. It also has a lower molecular weight than Gum Acacia Senegal and less emulsification capability. However, Gum Acacia Seyal has a greater ability to retain volatile agents (e.g., essential oils) than Gum Acacia Senegal.

In some instances, Gum Acacia Senegal and Gum Acacia Seyal can be obtained under the ENCAPCIA™ name and is available from Nexira, Inc., Route de Neufchatel, 76440 Serqueux, France. The ENCAPCIA™ has approximately 2.7 wt. % protein, has a higher molecular weight as compared to other acacia gums, and is a larger molecule in size because of its content of branched proteins and method for concentrating the proteins. The ENCAPCIA™ also has an approximately 1:1 ratio of Gum Acacia Senegal to Gum Acacia Seyal.

SPRAYGUM™ BA acacia gum is also available from Nexira, Inc. It is primarily a Seyal acacia gum having approximately 1.0 wt. % protein and a lower molecular weight.

The protein and carbohydrate components of the acacia gum are important for its functionality and provides acacia gum its advantageous emulsifying and stabilizing properties.

In some cases, the balance between the two are important to consider due to the tackiness, size of the spray dry material and stability of the product. The essential oil needs a good stable tight spray dry material to avoid loss and protect it from volatizing during the spray drying process.

While Gum Acacia Seyal can have less effective emulsification capabilities, it does have advantageous properties for retention of agents, particularly volatile agents such as essential oils. In contrast, Gum Acacia Senegal has highly effective emulsification capabilities while its retention capabilities, especially for volatile agents (e.g., essential oils) is lower. Consequently, a mixture of Gum Acacia Seyal and Gum Acacia Senegal could be advantageous to provide sufficient emulsification capability and sufficient retention of volatile agents in the emulsion.

The weight ratio of the Gum Acacia Senegal to the Gum Acacia Seyal can be from about 1:6 to about 6:1, from about 1:5 to about 5:1, from about 1:5 to 4:1, from about 1:5 to 3:1, from about 1:5 to 2:1, from about 1:5 to 1:1, from about 1:4 to 5:1, from about 1:3 to 5:1, from about 1:2 to 5:1, from about 1:1 to 5:1, from about 1:4 to 4:1, from about 1:3 to 3:1, from about 1:2 to 2:1, from about 1:2 to 1:1, from about 1:1 to 2:1, or about 1:1, based on the weight of each acacia gum.

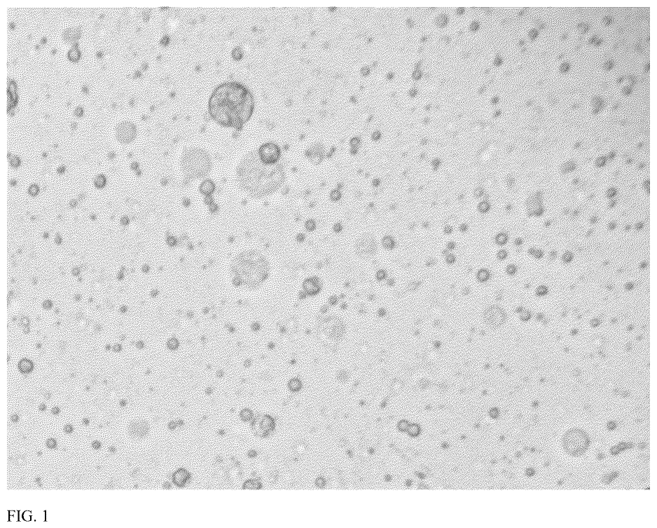

FIG. 1 shows a 40 times magnification of the spray dried particles resulting from the composition of about 50 wt. % acacia gum (i.e., SPRAYGUM™ BA), about 48 wt. % oil (i.e., blend of geraniol, clove bud oil, peppermint oil, thyme oil, cedarwood oil, lemongrass oil, and lauric acid) and about 2 wt. % ALCOLEC® S soy lecithin available from American Lecithin having about 39-45 wt. % phospholipid content. The particles are irregular indicating that the acacia gum with its hydrophobicity is present in the particles. The average particle size is approximately 600 nm.

Additionally, the spray dried particles can have a concentration of about 68 wt. % oil, about 30 wt. % acacia gum (Seyal, Senegal, or a combination thereof), and 2 wt. % soy lecithin (i.e., phospholipid).

An important parameter is the amount of acacia gum needed for the process while limiting the concentration in the spray dried composition. The concentration of the acacia gum in the emulsion prepared for the spray drying process is from about 5 wt. % to about 30 wt. %, from about 5 wt. % to about 25 wt. %, from about 5 wt. % to about 20 wt. %, from about 5 wt. % to about 15 wt. %, from about 10 wt. % to about 30 wt. %, from about 10 wt. % to about 25 wt. %, from about 10 wt. % to about 20 wt. %, from about 10 wt. % to about 15 wt. %, or about 15 wt. %, based on the total weight of the emulsion.

The compositions can have the polysaccharide polymer comprise from about 10 wt. % to about 40 wt. %, from about 10 wt. % to about 35 wt. %, from about 10 wt. % to about 30 wt. %, from about 20 wt. % to about 40 wt. %, from about 20 wt. % to about 35 wt. %, or from about 20 wt. % to about 30 wt. %, based on the total weight of the polymer, the medium chain fatty acids, and the active agent.

The compositions can have the medium chain fatty acid or salt or ester thereof is present in palm kernel oil, coconut oil, or a combination thereof.

The compositions can also have the medium chain fatty acid or salt or ester thereof comprise caproic acid, caproate, caprylic acid, caprylate, capric acid, caprate, lauric acid, laurate, or a combination thereof.

The compositions can have the medium chain fatty acid or salt or ester thereof comprise caprylic acid, caprylate, capric acid, caprate, or a combination thereof.

The compositions preferably have the medium chain fatty acid or salt or ester thereof comprise capric acid, caprate, or a combination thereof.

The compositions can also have the medium chain fatty acid or salt or ester thereof comprise a combination of palm kernel oil, coconut oil, or a combination of palm kernel oil and coconut oil and at least one of caproic acid, caproate, caprylic acid, caprylate, capric acid, caprate, lauric acid, laurate, or a combination thereof.

The compositions further have the medium chain fatty acid or salt or ester thereof comprise a combination of coconut oil and at least one of caproic acid, caproate, caprylic acid, caprylate, capric acid, caprate, lauric acid, laurate, or a combination thereof.

The compositions can have the medium chain fatty acid or salt or ester thereof comprise from about 25 wt. % to about 70 wt. %, from about 25 wt. % to about 65 wt. %, from about 30 wt. % to about 70 wt. %, from about 30 wt. % to about 65 wt. %, from about 35 wt. % to about 70 wt. %, or from about 35 wt. % to about 65 wt. % based on the total weight of the polymer, the medium chain fatty acids, and the active agent.

The compositions can also contain additional fatty acids or esters thereof including 2-monomyristin (i.e., glyceryl 2-myristate or 1.3-dihydroxypropan-2-yl tetradeconoate); decanoic acid, 1,2,3-propanetriyl ester; or a combination thereof.

The compositions can further have the active agent comprise an antibacterial, a color compound, a fragrance compound, an active pharmaceutical ingredient (API), an over-the-counter (OTC) ingredient, an antimicrobial, an antifungal, an antioxidant, a flavinoid, an essential oil, or a combination thereof.

The compositions can have the active agent comprise an EPA registered repellant.

The compositions can further have the active agent comprise N,N-diethyl-meta-toluamide (DEET), ethyl butylacetylaminopropionate (IR3535®), Picaridin, oil of lemon eucalyptus, undecan-2-one, or a combination thereof.

The compositions can also have the active agent comprise an insecticide, a herbicide, a rodenticide, a fungicide, or a combination thereof.

The compositions preferably have the active agent comprise an essential oil.

The compositions can have the essential oil comprise thymol oil, catnip oil, basil oil, geraniol oil, geranium oil, peppermint oil, rosemary oil, citronella oil, cedarwood oil, lemongrass oil, cinnamon oil, clove oil, vanillin, lavender oil, tea tree oil, lemon oil, eucalyptus oil, frankincense oil, chamomile oil, sandalwood oil, bergamot oil, sage oil, orange oil, corn oil, cornmint oil, cottonseed oil, garlic oil, linseed oil, sesame oil, soybean oil, spearmint oil, thyme oil, ginger oil, mandarin oil, or a combination thereof.

The compositions can have the active agent comprise from about 0.1 wt. % to about 25 wt. %, from about 0.1 wt. % to about 20 wt. %, from about 0.1 wt. % to about 15 wt. %, from about 0.1 wt. % to about 12 wt. %, from about 1 wt. % to about 25 wt. %, from about 1 wt. % to about 20 wt. %, from about 1 wt. % to about 15 wt. %, from about 1 wt. % to about 12 wt. %, from about 2 wt. % to about 15 wt. %, or from about 2 wt. % to about 12 wt. % based on the total weight of the polymer, the medium chain fatty acids, and the active agent.

The compositions described herein can further comprise a free essential oil that is not encapsulated by the polysaccharide polymer.

The compositions can also further comprise a surfactant.

The composition can have the surfactant be a cationic, an anionic, or a nonionic surfactant.

The compositions described herein can also have the surfactant comprise a stearic acid, a polyglycerin having 2-10 glycerine units, an alkyl sulfate, or a combination thereof.

The compositions also have the surfactant comprising polyglyceryl monostearate, sodium lauryl sulfate, or a combination thereof.

For the compositions described herein, the surfactant can have a concentration from about 0.1 wt. % to about 5 wt. %, from about 0.1 wt. % to about 4 wt. %, from about 0.1 wt. % to about 3 wt. %, from about 0.2 wt. % to about 5 wt. %, from about 0.2 wt. % to about 4 wt. %, or from about 0.2 wt. % to about 3 wt. %, based on the total weight of the polymer, the medium chain fatty acids, the active agent, and the surfactant.

The compositions can also have the ratio of the medium chain fatty acids to the active agent be from about 5:1 to about 30:1, from about 5:1 to about 25:1, from about 5:1 to about 20:1, from about 7:1 to about 30:1, from about 7:1 to about 25:1, from about 7:1 to about 20:1, from about 10:1 to about 30:1, from about 10:1 to about 25:1, or from about 5:1 to about 20:1.

The essential oil and free essential oil can be extracted from their natural source via any method known in the art. Examples include selective fractionation, solvent extraction, subcritical water extraction, and carbon dioxide extraction. Extraction can lead to the isolation of known actives of essential oils like monoterpenes such as carvacrol, p-cymene, linalool, alpha-terpinene, and thymol.

The composition can further comprise at least one additional polysaccharide coating. The weight ratio of the at least one polysaccharide to the at least one additional polysaccharide coating can be about 5:1 to 1:5.

### Dry Topical Compositions and Methods of Use

The present disclosure is further directed to a dry topical composition comprising a dry spray dried powder composition generally prepared using the spray drying method described herein for the compositions described above and combining the resulting spray dried powder with at least one carrier.

The carrier can comprise a fatty alcohol, a saturated fatty acid, medium-chain fatty acid (MCFA), a carboxylic acid, a lipid, a steroid, a wax, an alkane, a synthetic polymer, or a combination thereof.

The medium-chain fatty acid can comprise a C₈, C₁₀, or C₁₂ medium fatty acid. The medium-chain fatty acid can comprise dodecanoic acid, decanoic acid, octanoic acid, oleic acid, or a combination thereof.

The composition can further comprise an adjuvant selected from a pesticide, a fungicide, an antiviral, an antiparasitic, or a combination thereof. The adjuvant can be found within the polysaccharide coating, the zeolite particle, or a combination thereof.

Exemplary pesticides classified as minimum risk active ingredients under Section 25(b) FIFRA include Castor Oil, Cedarwood Oil, Chitosan, Cinnamon and Cinnamon Oil, Citric Acid, Citronella and Citronella Oil, Cloves and Clove Oil, Corn Gluten Meal, Corn Oil, Cornmint and Cornmint Oil, Cottonseed Oil, Dried Blood, Eugenol, Garlic and Garlic Oil, Geraniol, Geranium Oil, Lauryl Sulfate, Lemon grass Oil, Linseed Oil, Malic Acid, Peppermint and Peppermint Oil, 2-phenylethyl propionate, Potassium Sorbate, Putrescent Whole Egg Solids, Rosemary and Rosemary Oil, Sesame and Sesame Oil, Sodium Chloride, Sodium Lauryl Sulfate, Soybean Oil, Spearmint and Spearmint Oil, Thyme and Thyme Oil, White Pepper, Zinc Metal Strips, and combinations thereof. Several of these pesticides are essential oils.

Exemplary fungicide include Fluconazole, Oxiconazole, Terconazole, Clotrimazole, Anidulafungin, Griseofulvin, Caspofungin, Tolnaftate, Voriconazole, Butoconazole, Nystatin, Amphotericin B, Naftifine, Pentamidine, Haloprogin, Natamycin, Terbinafine, Sirolimus, Salicylic acid, Tioconazole, Ketoconazole, Cerulenin, Butenafine, Flucytosine, Miconazole, Econazole, Micafungin, Candicidin, Sertaconazole, Itraconazole, Ciclopirox, Chloroxine, Posaconazole, Nitroxoline, Sinefungin, Thymol, Radicicol, Benzoic acid, Salicylhydroxamic Acid, Myxothiazol, Lawsone, Bifonazole, Mevastatin, Bafilomycin A1, Sulconazole, Isoconazole, Hexetidine, Miltefosine, Efinaconazole, Tavaborole, Amorolfine, Cordycepin, Nikkomycin Z, Tolciclate, Flutrimazole, Fenticonazole, Pyrrolnitrin, Mepartricin, Omoconazole, Nifuratel, Wortmannin, Capric acid, Albaconazole, Isavuconazole, Ravuconazole, Abafungin, Isavuconazonium, Luliconazole, Pecilocin, Pentamycin, Basifungin, Pyrithione, Pyroquilon, Olorofim, Potassium iodate, Troclosene, Triacetin, Efungumab, Rezafungin, Levoketoconazole, Ibrexafungerp, Trichostatin A, Oteseconazole, Ajoene, VL-2397, and combinations thereof.

Exemplary antivirals include Abacavir, Acyclovir (Aciclovir), Adefovir, Amantadine, Ampligen, Amprenavir (Agenerase), Umifenovir (Arbidol), Atazanavir, Atripla (Efavirenz/emtricitabine/tenofovir), Efavirenz, Emtricitabine, Tenofovir disoproxil, Baloxavir marboxil (Xofluza), Biktarvy (Bictegravir/emtricitabine/tenofovir alafenamide), Bictegravir, Emtricitabine, Tenofovir alafenamide, Boceprevir, Bulevirtide, Cidofovir, Cobicistat (Tybost), Combivir (Lamivudine/Zidovudine), Lamivudine, Zidovudine, Daclatasvir (Daklinza), Darunavir, Delavirdine, Descovy (Emtricitabine/tenofovir alafenamide), Emtricitabine, Tenofovir alafenamide, Didanosine, Docosanol, Dolutegravir, Doravirine (Pifeltro), Edoxudine, Efavirenz, Elvitegravir, Emtricitabine, Enfuvirtide, Ensitrelvir, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Inosine pranobex, Indinavir, Lamivudine, Letermovir (Prevymis), Lopinavir, Loviride, Maraviroc, Methisazone, Molnupiravir, Moroxydine, Nelfinavir, Nirmatrelvir/ritonavir (Paxlovid), Nevirapine, Nitazoxanide, Norvir, Oseltamivir (Tamiflu), Peramivir (Rapivab), Penciclovir, Pleconaril, Podophyllotoxin, Raltegravir, Remdesivir, Ribavirin, Rilpivirine (Edurant), Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tipranavir, Trifluridine, Trizivir, Tromantadine, Truvada, Umifenovir, Valaciclovir, Valganciclovir, Vicriviroc, Vidarabine, Zalcitabine, Zanamivir, Zidovudine, and combinations thereof.

Exemplary antiparasitics include Ivermectin, Pyrimethamine, Dapsone, Doxycycline, Mefloquine, Sulfadiazine, Quinine, Nitazoxanide, Albendazole, Piperazine, Amodiaquine, Furazolidone, Mebendazole, Sulfametopyrazine, Amphotericin B, Diethylcarbamazine, Thiabendazole, Pentamidine, Levamisole, Quinidine, Tinidazole, Metronidazole, Praziquantel, Primaquine, Oxamniquine, Atovaquone, Proguanil, Halofantrine, Posaconazole, Sulfadoxine, Paromomycin, Hydroxychloroquine, Sinefungin, 5′-S-methyl-5′-thioadenosine, Fumagillin, Benzimidazole, Diminazene, Radicicol, Salicylhydroxamic Acid, Geneticin, Bithionol, Halofuginone, Oxibendazole, Lucanthone, Andrographolide, Spiramycin, Eflornithine, Lonidamine, Tafenoquine, Lumefantrine, Niclosamide, Pyrvinium, Anisomycin, Puromycin, Diloxanide, Flubendazole, Miltefosine, Chlortetracycline, Diiodohydroxyquinoline, Artesunate, Pyrantel, Hexylresorcinol, Amprolium, Cambendazole, Clorsulon, Coumaphos, Decoquinate, Dichlorophen, Dichlorvos, Diclazuril, Doramectin, Eprinomectin, Febantel, Fenbendazole, Lasalocid, Monensin, Moxidectin, Narasin, Nicarbazin, Oxfendazole, Phenothiazine, Roxarsone, Selamectin, Sulfaquinoxaline, Metrifonate, Zoalene, Bunamidine, Dithiazanine, Hygromycin B, Imidocarb, Milbemycin oxime, Robenidine, Salinomycin, Artenimol, Misonidazole, Nifurtimox, Benznidazole, Nimorazole, Triclabendazole, Chlorproguanil, Oltipraz, Mizoribine, Secnidazole, Melarsoprol, Pyronaridine, Carbendazim, Ornidazole, Etofamide, Propamidine, Mepartricin, Niridazole, Oxantel, Nifuratel, Meglumine antimoniate, Albendazole oxide, Piperaquine, Stibophen, Bephenium, Ciclobendazole, Desaspidin, Hachimycin, Chloroquine, Quinacrine, Suramin, Sodium stibogluconate, Artemether, Chloroxylenol, Emetine, Artemotil, Hycanthone, Tetrandrine, Diloxanide furoate, Betulinic Acid, Cycloguanil, Pafuramidine, Emodepside, Artefenomel, Antimony potassium tartrate, Homidium, Plumbagin, Allopurinol riboside, Lotilaner, Psoralen, Santonin, and combinations thereof.

The compositions can also comprise retinoids. For example, the retinoid can be a derivative of vitamin A, such as retinol and retinyl palmitate. These compounds are oil-soluble and are considered the gold standard for anti-aging. They work by increasing the rate of cell turnover and boosting collagen production, which helps to minimize the appearance of fine lines, wrinkles, and dark spots.

The compositions can also comprise ascorbyl tetraisopalmitate. This is a highly stable and effective oil-soluble form of vitamin C. Unlike its water-soluble counterpart, it easily penetrates the skin to deliver powerful antioxidant benefits, protecting against UV damage, and stimulating collagen synthesis.

The compositions can also comprise bakuchiol. Bakuchiol is a plant-based alternative to retinol and offers similar anti-aging benefits without the potential irritation often associated with retinoids. It helps to increase cell turnover and improve firmness and elasticity.

The compositions can also comprise Coenzyme Q10 (Ubiquinone). As a potent antioxidant, CoQ10 protects against free radical damage and helps to reduce the appearance of wrinkles caused by photoaging. It is important for cellular energy production and is easily incorporated into oil-based formulas.

Also, the compositions can comprise nutrient-rich botanical oils. The botanical oils can comprise rosehip oil, sea buckthorn oil, pomegranate seed oil, jojoba oil, argan oil, vitamin E (tocopherol) oil, or a combination thereof.

Rosehip oil is rich in essential fatty acids, antioxidants, and a naturally occurring form of vitamin A (retinoic acid). This combination helps to stimulate cell regeneration, boost collagen production, and improve skin tone and texture.

Sea buckthorn oil contains over 190 bioactive compounds, including antioxidants, vitamins, and omegas 3, 6, 7, and 9, sea buckthorn oil is a nutritional powerhouse for the skin. It helps to boost collagen production, improve skin elasticity, and protect against free radical damage.

Pomegranate seed oil is a potent oil packed with powerful antioxidants, such as punicic acid and ellagic acid. It offers anti-inflammatory and antioxidant benefits that protect the skin and support collagen synthesis.

Jojoba oil is technically a liquid wax and has a molecular structure that closely mimics the skin's natural sebum, making it highly compatible and non-comedogenic. It provides a protective moisture barrier while delivering antioxidants and vitamin E to defend against oxidative damage.

Argan oil is often called “liquid gold” and is rich in antioxidants, vitamin E, and essential fatty acids that deeply moisturize and improve skin elasticity. It helps to repair the skin's barrier function and decrease the appearance of fine lines.

Vitamin E (Tocopherol) oil is a powerful, fat-soluble antioxidant and is known for its ability to protect skin cells from oxidative stress and free radical damage caused by UV exposure. It also has moisturizing and healing properties that help strengthen the skin's barrier.

The composition can further comprise at least one hard oil, wax, or combination thereof. In certain embodiments, the wax is carnauba wax.

The composition can further comprise an oil. The oil can comprise palm oil, castor oil, hydrogenated palm oil, hydrogenated castor oil, or a combination thereof.

The composition can further comprise a thickener. The thickener can comprise a wax, a fatty acid, a fatty alcohol, or a combination thereof. The fatty acid can be lauric acid.

Lauric acid is a dual purpose ingredient; it can act as a thickener and an insect repellent.

The composition can further comprise a mineral powder selected from calcium carbonate, silicon dioxide, sodium carbonate, sodium bicarbonate, or a combination thereof.

The topical composition can be an insect repellent.

The composition can be categorized as minimum risk under Section 25(b) of FIFRA (Federal Insecticide, Fungicide Rodenticide Act).

The topical composition can be a sunscreen, insect repellent, or combination sunscreen insect repellent.

The composition can be categorized as minimum risk under Section 25(b) of FIFRA (Federal Insecticide, Fungicide Rodenticide Act).

The present disclosure is further directed to a dry sunscreen composition comprising the dry spray dried powder composition as disclosed herein and at least one carrier.

The dry sunscreen composition can further comprise a mineral sunscreen ingredient such as zinc oxide, zinc stearate, titanium dioxide, or a combination thereof.

The dry sunscreen composition can also further comprise a chemical sunscreen ingredient. The chemical sunscreen ingredient can be selected from the group consisting of avobenzone, homosalate, octisalate, butyl octylsalicylate, oxybenzone, octinoxate, octocrylene, Padimate O, bemotrizinol (BEMT), and combinations thereof.

The chemical or mineral sunscreen ingredient can be any chemical or mineral sunscreen ingredient known in the art. The chemical or mineral sunscreen ingredient can protect against radiation between about 280 and about 400 nm (i.e. blocking UVA/UVB radiation). The compound class can be conjugated aromatics.

The dry sunscreen composition can have an SPF value of about 1 to about 100, about 2 to about 50, about 3 to about 35, about 4 to about 49, or about 5 to about 75.

The present disclosure is also directed to a method of blocking sun exposure of a subject, the method comprising administering the dry sunscreen composition as disclosed herein to the subject.

The composition can further comprise a molecular sieve.

This disclosure describes a dry spray dried powder composition comprising: a) molecular sieves impregnated with at least one essential oil; and b) at least one free essential oil; wherein the at least one free essential oil and molecular sieves are coated or partially coated with at least one polysaccharide.

The coating can be generally spherical but irregular. The thickness of the coating is determined by the size of the apparatus tip used to make the composition. Perforations and/or folds can exist within the coating. In certain cases, some of the molecular sieves protrude outside the coating.

Molecular sieves are materials with an interior network of pores about 2 nm in diameter or less. In certain instances, the pores are about 10 angstroms in diameter or less. Examples include aluminosilicates such as zeolites, activated charcoal, and silica gel. Some clays and porous glass are also molecular sieves. Molecular sieves can function as adsorbents and take up compounds that can fit within their pore sizes. They have many laboratory, industrial, and commercial applications. For example, they are often used in laundry detergents as water softeners. They can be used as desiccants and catalysts. They are also used in size exclusion chromatography and filters.

The molecular sieves can comprise or consist of zeolite particles. The zeolite particles can be made from natural or synthetic zeolite. The zeolite particles can be analcime, chabazite, clinoptilolite, heulandite, natrolite, phillipsite, stilbite, or a combination thereof. In particular, the zeolite particles are clinoptilolite. Commonly available synthetic zeolites are 3A, 4A, and 5A sieves, which are named for their effective pore size in angstroms, and 13X sieves.

Each zeolite nanoparticle can be about 200 nm to about 90 microns in size, about 1 micron to about 90 microns in size, about 1 micron to about 45 microns in size, about 1 micron to about 30 microns in size, about 1 micron to about 10 microns in size, about 10 microns to about 30 microns in size, or about 10 microns to about 15 microns in size. The average pore size of the zeolite particles can be about 1.0 angstrom to about 10.0 angstroms, about 4.0 angstroms to about 7.0 angstroms, or about 5.0 angstroms to about 6.0 angstroms.

In some cases, the zeolite nanoparticles are made the correct size by milling or shearing.

The zeolite particles can comprise from about 71.7% Si to about 91.7% Si, from about 5% Al to about 15% Al, from about 4% K to about 10% K, from about 0.5% Fe to about 1.5% Fe, from about 0.5% Ca to about 1.5% Ca, from about 0.25% Mg to about 1.25% Mg, and from about 0.75% Na to about 1.75% Na.

Further, the zeolite particles can be derived from about 67.04% SiO₂ to about 87.04% SiO₂, from about 7% Al₂O₃ to about 17% Al₂O₃, from about 1% K₂O to about 10% K₂O, from about 0.5% FeO to about 1.5% FeO, from about 0.5% CaO to about 1.5% CaO, from about 0.2% MgO to about 1.2% MgO, and from about 1% Na₂O to about 2% Na₂O.

In particular, the zeolite particles can comprise about 81.7% Si, 8.01% Al, 6.04% K, 1.145% Fe, 1.2% Ca, 0.725% Mg, and 1.18% Na. The zeolite particles can be derived from about 77.04% SiO₂, 13.2% Al₂O₃, 6.42% K₂O, 0.66% FeO, 0.75% CaO, 0.53% MgO, and 1.4% Na₂O.

The ratio of zeolite to total essential oil in the composition can be from about 10:1 to about 1:2, from about 9:1 to about 1:2 from about 8:1 to about 1:2, from about 7:1 to about 1:2, from about 6:1 to about 1:2, from about 5:1 to about 1:2, from about 4:1 to about 1:2, from about 3:1 to about 1:2, or from about 2:1 to about 1:2. Preferably, the ratio of zeolite to total essential oil in the composition can be about 2:1.

The ratio of zeolite to free essential oil in the composition can be from about 5:1 to about 1:4, from about 4:1 to about 1:4, from about 3:1 to about 1:4, from about 2:1 to about 1:4, from about 1:1 to about 1:4, from about 5:1 to about 1:3, from about 4:1 to about 1:3, from about 3:1 to about 1:3, from about 2:1 to about 1:3, from about 1:1 to about 1:3, from about 5:1 to about 1:2, from about 4:1 to about 1:2, from about 3:1 to about 1:2, from about 2:1 to about 1:2, from about 1:1 to about 1:2, from about 5:1 to about 1:1, from about 4:1 to about 1:1, from about 3:1 to about 1:1, or from about 2:1 to about 1:1. Preferably, the ratio of zeolite to free essential oil in the composition can be about 1:1.

In some instances, the zeolite particles, at least one free essential oil, and polysaccharide form an agglomerate with a longest dimension of about 100 microns to about 10 mm, about 300 microns to about 5 mm, or about 500 microns to about 2 mm.

### Spray-Dried Compositions

The compositions described above can generally be used in the spray drying process. In some cases, additional components are added to the compositions to facilitate spray drying or modify the properties of the compositions.

Preferably, the spray dry composition before and after the composition undergoes the spray drying process is hydrophobic. The hydrophobicity is affected by adding oils (including the medium chain fatty acids detailed above), lipids, phospholipids, hydrophobic polymers or crosslinkers to the composition that will undergo the spray drying process.

In addition, crosslinking of the polymer component (e.g., crosslinking acacia gum with a different polysaccharide like glutaraldehyde) can induce encapsulation and hydrophobicity.

Adding a surfactant to the polymer component (e.g., acacia gum, carboxy methyl cellulose and the like) where its hydrophobic tail interacts with the surface of the polymer can increase hydrophobicity of the spray dry composition.

Oils or other highly hydrophobic components can be added at different times during the spray drying process to affect the hydrophobicity of the spray dry particles being made. For example, an oil or hydrophobic compound could be added to the initial composition with the polymer component that is spray dried to make a particle (e.g., in the first pass) and then the oil of hydrophobic compound can also be added to a secondary chamber or double pass system where the oil is added to the spray dried particles without the addition of the polymer component.

A phospholipid is preferably added to the oil phase at a concentration from about 0.1 wt. % to about 10 wt. %, from about 0.1 wt. % to about 9 wt. %, from about 0.1 wt. % to about 8 wt. %, from about 0.1 wt. % to about 7 wt. %, from about 0.1 wt. % to about 6 wt. %, from about 0.1 wt. % to about 5 wt. %, from about 0.1 wt. % to about 4 wt. %, from about 0.1 wt. % to about 3 wt. %, from about 0.1 wt. % to about 2 wt. %, from about 0.1 wt. % to about 1 wt. %, from about 0.25 wt. % to about 10 wt. %, from about 0.25 wt. % to about 9 wt. %, from about 0.25 wt. % to about 8 wt. %, from about 0.25 wt. % to about 7 wt. %, from about 0.25 wt. % to about 6 wt. %, from about 0.25 wt. % to about 5 wt. %, from about 0.25 wt. % to about 4 wt. %, from about 0.25 wt. % to about 3 wt. %, from about 0.25 wt. % to about 2 wt. %, from about 0.25 wt. % to about 1 wt. %, from about 0.5 wt. % to about 10 wt. %, from about 0.5 wt. % to about 9 wt. %, from about 0.5 wt. % to about 8 wt. %, from about 0.5 wt. % to about 7 wt. %, from about 0.5 wt. % to about 6 wt. %, from about 0.5 wt. % to about 5 wt. %, from about 0.5 wt. % to about 4 wt. %, from about 0.5 wt. % to about 3 wt. %, from about 0.5 wt. % to about 2 wt. %, from about 0.5 wt. % to about 1 wt. %, based on the total weight of the oil phase. Preferably, about 2 wt. % of the phospholipid (i.e., soy lecithin) is added to the oil phase.

The phospholipid can be present in the spray dried composition at a concentration from about 0.2 wt. % to about 6 wt. %, from about 0.2 wt. % to about 5 wt. %, from about 0.2 wt. % to about 4 wt. %, from about 0.2 wt. % to about 3 wt. %, from about 0.2 wt. % to about 2 wt. %, from about 0.2 wt. % to about 1 wt. %, from about 0.3 wt. % to about 6 wt. %, from about 0.3 wt. % to about 5 wt. %, from about 0.3 wt. % to about 4 wt. %, from about 0.3 wt. % to about 3 wt. %, from about 0.3 wt. % to about 2 wt. %, from about 0.3 wt. % to about 1 wt. %, based on the total weight of the spray dried composition (total weight of essential oil, phospholipid, zeolite (if present), and acacia gum or other polymer).

The phospholipid can comprise a lecithin. Preferably, the lecithin is an enriched soy lecithin, sunflower lecithin, or egg lecithin.

Additionally, the lecithin can be a fractionation of lecithin having a particular concentration of phosphatidylcholine or that provides higher levels of phosphatidylcholine. The lecithin can be sourced from American Lecithin Company in Oxford, Connecticut.

Preferably, the lecithin is ALCOLEC® S, which is a liquid soy lecithin available commercially from American Lecithin Company in Oxford, Connecticut having a typical concentration of 14 wt. % phosphatidyl choline, from 39 wt. % to 45 wt. % phospholipids (e.g., phosphatidylcholine, phosphatidylethanolamine, phosphatidylinositol, and phosphatidic acid, among others), and a hydrophilic/lipophilic balance (HLB) of 4.

Additionally, other ALCOLEC® lecithins can be used including ALCOLEC® BS (fluid having 14 wt. % phosphatidylcholine), ALCOLEC® BS (fluid having 14 wt. % phosphatidylcholine), ALCOLEC® XTRA-A (fluid having 14 wt. % phosphatidylcholine), ALCOLEC® HL (fluid having 14 wt. % phosphatidylcholine), ALCOLEC® HR (fluid having 14 wt. % phosphatidylcholine), ALCOLEC® F-100 (powder having 24 wt. % phosphatidylcholine), ALCOLEC® FF-100 (fine powder having 24 wt. % phosphatidylcholine), ALCOLEC® H-20 (fine powder having 20 wt. % phosphatidylcholine), ALCOLEC® SGU (fluid having 14 wt. % phosphatidylcholine), ALCOLEC® Aquasperse A (fluid having 10 wt. % phosphatidylcholine), ALCOLEC® LV-30 (fluid having 11 wt. % phosphatidylcholine), ALCOLEC® 40P PCR Neg (powder having 40 wt. % phosphatidylcholine), ALCOLEC® 40P Phsphal Series (fluid having from 34 wt. % to 72 wt. % wt. % phosphatidylcholine), ALCOLEC® LEM (fluid having 14 wt. % phosphatidylcholine), ALCOLEC® EM (powder having 24 wt. % phosphatidylcholine), LIPOID© LPC 80 (agglomerate having 80 wt. % phosphatidylcholine), PHOSAL® 75 SA (27 wt. % to 78 wt. % phosphatidylcholine dissolved in a carrier system consisting primarily of safflower oil), PHOSAL® 53 MCT (Minimum 53 wt. % phosphatidylcholine solubilized in a carrier system comprised primarily of medium chain triglycerides), PHOSAL® 50 SA+ (Soy) (Minimum 50 wt. % phosphatidylcholine solubilized in a carrier system comprised primarily of safflower oil. Aid for the solubilization of lipophilic actives), PHOSAL® H 50 (50 wt. % sunflower phosphatidylcholine in sunflower oil), LIPOID® E 80 (fractionated egg lecithin containing at least 90 wt. % phospholipids), LIPOID® H 65 (agglomerate, 60 wt. % phosphatidylcholine (non-GMO)), LIPOID® H 85 (agglomerate, 85 wt. % phosphatidylcholine (non-GMO)), LIPOID® H 100 (agglomerate, 95 wt. % phosphatidylcholine (non-GMO)), LIPOID® H 100-3 (hydrogenated, 95 wt. % phosphatidylcholine (non-GMO)), LIPOID® P 45 (agglomerate, 45 wt. % phosphatidylcholine (non-GMO)), LIPOID® S 45 (agglomerate, 45 wt. % phosphatidylcholine (PCR negative)), LIPOID® P 75 (agglomerate, 70 wt. % phosphatidylcholine (non-GMO)), LIPOID® S 75 (agglomerate, 70 wt. % phosphatidylcholine (PCR negative)), PHOSAL® 35 SB (phospholipid fraction with typically 35 wt. % phosphatidylcholine and other phospholipids in a sunflower oil based carrier system), PHOSAL® 40 IP (all-natural, non-GMO phospholipid fraction from soybean with typically 40 wt. % phosphatidylcholine and other phospholipids in a sunflower oil based carrier system), or PHOSAL® 75 SA (72 wt. % to 78 wt. % phosphatidylcholine dissolved in a carrier system consisting primarily of safflower oil).

Further, the spray drying parameters can affect the hydrophobicity of the resulting spray dried particles. For example, the temperature and nozzle pressure can affect the hydrophobicity of the resulting spry dried particle. In general, lower temperatures and higher nozzle pressures can increase hydrophobicity of the resulting particles. The airflow and feed solution (as discussed above) are also considered to get the desired hydrophobicity.

The coating can be generally spherical but irregular. The thickness of the coating is determined by the size of the apparatus tip used to make the composition. Perforations and/or folds can exist within the coating. In certain cases, some of the molecular sieves protrude outside the coating.

The present disclosure is further directed to a dry insect repellent composition comprising the dry spray dried powder composition as disclosed herein and at least one carrier. The insect repellent composition can further comprise DEET, Icaridin (Picaridin) IR3535, KBR 3023, a terpene, an astringent, or a combination thereof.

The present disclosure is also directed to a method of repelling insects from a subject, the method comprising administering the insect repellent composition as disclosed herein to or near a subject.

The subject can be a mammal, preferably a human. The subject can be a mammal, such as a human or livestock animal such as cow, horse, or pig.

The present disclosure is further directed to a method of repelling insects from livestock, the method comprising: a) generating a spray composition by combining the composition described herein of the zeolite particles, at least one free essential oil, and polysaccharide that form an agglomerate with water; and b) spraying the livestock with the spray composition. The method can be completed within one hour, two hours, six hours, or ten hours. The livestock can be selected from the group consisting of cows, pigs, and horses.

The dry topical compositions or the dry insect repellant compositions as disclosed here can have less than about 10 wt. %, less than about 9 wt. %, less than about 8 wt. %, less than about 7 wt. %, less than about 6 wt. %, less than about 5 wt. %, less than about 4 wt. %, less than about 3 wt. %, less than about 2 wt. %, or less moisture.

The essential oil can be released from the composition when the subject perspires.

The compositions disclosed herein can also be emulsified in water for a composition containing up to about 10% wt. % water. This is preferably done as close as possible to the time of application to reduce premature release of the at least one essential oil.

The compositions disclosed herein can be formulated as several different types of emulsions, including oil, oil in water, water in oil, and water in oil in water emulsion types.

Representative formulations for the spray dried encapsulation compositions are detailed below. The first table provides general ranges for the dry spray dried encapsulation compositions and the second table details representative specific formulations for encapsulation spray dry formations.

| Component | Weight Percent (%) |
|---|---|
| Active Ingredient | 0.1-60 |
| Free Oil | 0.1-99.99 |
| Zeolite | 0-65 |
| MCT Fractionated | 0-80 |
| Coconut Oil | 0.1-80 |
| Palm Kernel Oil | 40-99.9 |
| Surfactant | 0-35 |
| Emulsifier | 0-35 |
| Polymer Component 1 | 0-30 |
| Polymer Component 2 | 0-30 |
| Wax | 0-97 |
| Core Modifier | 0-25 |

| | Encapsulation Spray Dry Formulations | | |
|---|---|---|---|
| | Encapsu- | Encapsu- | Encapsu- |
| | lation | lation | lation |
| | Formu- | Formu- | Formu- |
| | lation # 1 | lation # 2 | lation # 3 |
| Water | | | |
| Carageenan Kappa | | 5 | |
| Polymer | | | |
| Carageenan Alpha | | | |
| Polymer | | | |
| Carageenan Iota Polymer | | | |
| Chitosan Polymer | | | |
| Acacia Gum Polymer | 29.8 | 19 | 30 |
| Wax or Structurant/Fatty | 3 | | |
| alcohol or Acid/Quat | | | |
| (e.g., Carnauba Wax) | | | |
| Glycerin | | | |
| CarboxyMethyl Celulose | | | |
| Polymer | | | |
| Gelatin | | | |
| Coconut Oil(56-62%) | 47 | 37.3 | 5 |
| MCT | | | |
| MCT OIL | 12.45 | 25 | 61 |
| Cedarwood Oil | 0.05 | 10 | |
| Cinnamon Oil | 0.3 | | |
| Lemon Eucalyptus Oil | 1 | 0.5 | 2 |
| Peppermint Oil | 4 | | 0.5 |
| Lavendar Oil | 0.3 | | |
| Geranium Oil | 1 | 0.5 | |
| Citronella Oil | 0.9 | | |
| Geraniol | | | |
| Preservative | | | |
| Surfactant Sodium | 0.2 | 2.7 | 1.5 |
| Lauryl Sulfate Powder | | | |
| Total | 100 | 100 | 100 |

Additionally, a solid anhydrous formulation can be prepared and results in the formulations below.

| Component | Weight Percent (%) |
|---|---|
| Active Ingredient | 0.1-65 |
| Free Oil | 0.5-35 |
| Zeolite | 0-65 |
| MCT Fractionated | 0-80 |
| Coconut Oil | 0-80 |
| Surfactant | 0-35 |
| Wax | 1-99.9 |
| Core Modifier | 0-95 |

### Dissolution and Emulsion Compositions

The compositions can also be prepared as the encapsulated compositions are suspended or dissolved in an aqueous solvent (e.g., water) or the encapsulated compositions are prepared in an emulsion with an oil phase and an aqueous phase.

The dry spray-dried encapsulation compositions can be dissolved in water or another aqueous solvent and the following tables detail the general compositions and representative formulations.

| Component | Weight Percent (%) |
|---|---|
| Active Ingredient | 0.1-15 |
| Free Oil | 0-60 |
| Zeolite | 0-50 |
| MCT Fractionated | 0-80 |
| Coconut Oil | 0.1-80 |
| Emulsifier | 0-10 |
| Polymer Component 1 | 25 |
| Polymer Component 2 | 5 |

Representative formulations of the dissolution of the encapsulated spray dried composition being released into the water.

| | Disolution of Encap being released | | |
|---|---|---|---|
| | from polymer | | |
| | Formu- | Formu- | Formu- |
| | la#1 | la#2 | la#3 |
| Water | 50 | 30 | 60 |
| Carageenan Kappa Polymer | 0.5 | 2.5 | |
| Carageenan Alpha Polymer | | | 0.2 |
| Carageenan Iota Polymer | | | 0.3 |
| Chitosan Polymer | | | |
| Acacia Gum Polymer | 11.92 | 9.5 | 6 |
| Wax or Structurant/ | 2 | | |
| Fatty alcohol | | | |
| or Acid/Quat [symbol] | | | |
| Glycerin | 5 | 8 | 2 |
| CarboxyMethyl Celulose | 0.5 | | |
| Polymer Gelatin | | 0.2 | |
| Coconut Oil(56-62%) | 18.8 | 18.65 | 1 |
| MCT | | | |
| MCT OIL | 4.98 | 12.5 | 12.2 |
| Cedarwood Oil | 4.02 | 5 | 1.2 |
| Cinnamon Oil | 0.12 | 1.2 | 5.1 |
| Lemon Eucalyptus Oil | 0.4 | 0.25 | 3 |
| Peppermint Oil | 0.8 | 4 | 0.1 |
| Lavendar Oil | 0.12 | 1.1 | |
| Geranium Oil | 0.4 | 0.25 | 6.8 |
| Citronella Oil | 0.36 | 4 | |
| Geraniol | | 1 | 0.6 |
| Preservative | 6 | 0.5 | 1.2 |
| Surfactant Sodium | 0.08 | 1.35 | 0.3 |
| Lauryl Sulfate Powder | | | |
| TOTAL | 106 | 100 | 100 |

[symbol]

indicates data missing or illegible when filed

The emulsion formulations generally can have the following ranges of components.

| Component | Weight Percent (%) |
|---|---|
| Active Ingredient | 1-10 |
| Free Oil in Encapsulated Composition | 0-60 |
| Free Oil in Oil Phase | 0-97 |
| Zeolite | 0-65 |
| MCT Fractionated | 0-99.9 |
| Coconut Oil | 0.1-80 |
| Humectant | 0-30 |
| Emulsifier | 0-10 |
| Polymer Component 1 | 0-50 |
| Polymer Component 2 | 0-50 |

Representative formulations for the emulsions follow.

| | Emulsion Formulations without Encap Form | | | |
|---|---|---|---|---|
| | Formu- | Formu- | Formu- | Formu- |
| | lation | lation | lation | lation |
| | #1 | #2 | #3 | #4 |
| Water | 38.71 | 51.2 | 10 | 75 |
| Carageenan Kappa Polymer | | | | |
| Carageenan Alpha Polymer | | 5 | | |
| Carageenan Iota Polymer | | | 0.5 | |
| Chitosan Polymer | | | | |
| Acacia Gum Polymer | 10 | 2 | 5 | 3 |
| Wax or Structurant/Fatty | | 0.5 | | |
| alcohol or Acid/Quat | | | | |
| (e.g., Soy Wax) | | | | |
| Glycerin | 5 | 2 | | |
| CarboxyMethyl Celulose | 0.9 | | 0.2 | |
| Polymer Gelatin | | | | |
| Extra Virgin Oil Coconut | 38 | 15 | 65 | |
| Oil(56-62%) MCT | | | | |
| MCT OIL | | 17 | | 10 |
| Cedarwood Oil | | | | |
| Cinnamon Oil | 1 | | | |
| Lemon Eucalyptus Oil | | | | |
| Peppermint Oil | | 0.7 | 3 | 8 |
| Lavender Oil | | 0.7 | | |
| Geranium Oil | 0.7 | | | |
| Citronella Oil | | 0.1 | 4 | |
| Geraniol | 1.3 | | 2.8 | |
| Preservative Other | | | | |
| Surfactant Sodium Lauryl | 1.89 | 0.2 | 2.5 | |
| Sulfate Powder | | | | |
| Thyme Oil | | 1.3 | | |
| Cetearyl Glucoside | | | | 4 |
| Glyceryl Monostearate | 2 | 2 | 5 | |
| Glyceryl Monooleate | | 2 | 2 | |
| Sodium Citrate | 0.5 | 0.8 | | |
| Total | 100 | 100.5 | 100 | 100 |

### Oil Formulations

Additionally, the encapsulated spray dried compositions can be incorporated into oil and stick formulations. For example, the oil formulation can comprise from about 20 wt. % to about 95 wt. % of one or more medium chain fatty acids or salts or esters thereof having 6-12 carbon atoms; and one or more active agents.

In some oil formulations, the active agent and medium chain fatty acid or salt or ester thereof can be coated or partially coated with a polymer. When the active agent and medium chain fatty acid are coated or partially coated with the polymer, the polymer can be one of the polymers detailed above in connection with the compositions.

In the oil formulations, preferably, the active agent is an essential oil and the essential oil can be any of the essential oils listed above in connection with the compositions described.

Where the oil formulation includes the encapsulated formulation #1, the zeolite component can be any of the zeolites described herein.

The following table generally describes the ranges of components present in the various formulations.

| Component | Weight Percent (%) |
|---|---|
| Active Agent | 0-65 |
| Free Oil | 0.5-25 |
| MCT Fractionated | 75-99.5 |
| Core Modifier | 0-40 |

Representative oil formulations are detailed in the table below.

| | Oil | | Stick | Stick | | Oil |
|---|---|---|---|---|---|---|
| | Formula | Oil | Formula | Formula | StickFormula | Formula |
| Anhydrous Oil Formulations | #1 | Formula#2 | #3 | #4 | #5 | #5 |
| Encap Formulation #1 | 15 | | 40 | 30 | | |
| Wax or Structurant/Fatty | | | 15 | 14 | 17 | |
| alcohol or Acid/Quat | | | | | | |
| (e.g., Carnuba) | | | | | | |
| Extra Virgin Oil Coconut | 84 | 20 | 38 | 15 | 80 | 98 |
| Oil(56-62%) MCT | | | | | | |
| MCT OIL | | 78.5 | | 36 | | |
| Cedarwood Oil | 0.2 | | | | | |
| Cinnamon Oil | | 0.4 | | 4 | 1 | 0.25 |
| Lemon Eucalyptus Oil | 0.6 | 0.1 | 2 | | | 1 |
| Peppermint Oil | | | 5 | | 1.4 | 0.75 |
| Lavender Oil | | 0.1 | | 0.25 | | |
| Geranium Oil | | 0.2 | | 0.5 | 0.6 | |
| Citronella Oil | 0.2 | 0.5 | | | | |
| Geraniol | | | | 0.25 | | |
| Thyme Oil | | 0.2 | | | | |
| Total | 100 | 100 | 100 | 100 | 100 | 100 |

### Method of Making

The present disclosure is further directed to a method of producing a dry powder composition, the method comprising: combining an essential oil with a thickener, and a hydrogenated oil to generate an essential oil particles composition; heating and shearing the essential oil particle composition; and combining the essential oil-coated particle composition with a polysaccharide composition comprising at least one polysaccharide and spray drying the combination to generate the dry powder composition.

The present disclosure is further directed to a method of producing a dry powder composition, the method comprising: drying zeolite particles at 70% or less humidity; combining the zeolite particles with an essential oil composition comprising a thickener, at least one essential oil, and a hydrogenated oil to generate an essential oil-zeolite particles composition; heating and shearing the essential oil-zeolite particle composition; and combining the essential oil-coated zeolite particle composition with a polysaccharide composition comprising at least one polysaccharide and preferably a phospholipid (i.e., lecithin) and spray drying the combination to generate the dry powder composition.

The spray drying process can have a formulation comprising a total to about 80 wt. % zeolite plus oil with the remainder of the dry phase being an anionic polymer and phospholipid.

To make the composition that is fed into the spray dryer, about 40 wt. % of the zeolite and oil composition is combined with about 60 wt. % water and the combination is spray dried.

The emulsion formed is preferably of a size from about 600 nm to 5 microns in one dimension in order to provide the most efficient encapsulation process.

For the spray dry settings, the inlet temperabout is preferably 110° C. and the outlet temperature is about 60° C.

The spray drying is performed at about 1 to 4 bars pressure, preferably, the pressure is 3 bar.

In this spray drying process, the emulsion can be formed using phospholipid (lecithin) and acacia.

The thickener can comprise a wax, a fatty alcohol, a fatty acid, or a combination thereof.

The fatty alcohol can be a C₈ to C₁₆ fatty alcohol.

The fatty acid can be a C₈ to C₁₆ fatty acid; preferably, the fatty acid comprises lauric acid.

The emulsion prepared to be used in the spray drying process can have an aqueous phase and an oil phase. The aqueous phase can be prepared to be from about 60 wt. % to about 75 wt. %, from about 65 wt. % to about 75 wt. %, from about 70 wt. % to about 75 wt. %, from about 71 wt. % to about 75 wt. %, from about 71 wt. % to about 74 wt. %, from about 71 wt. % to about 73 wt. %, or about 72 wt. % of the total emulsion weight of the aqueous phase and the oil phase.

Further the aqueous phase can have a water concentration from about 50 wt. % to about 70 wt. %, from about 55 wt. % to about 70 wt. %, from about 57 wt. % to about 68 wt. %, from about 57 wt. % to about 65 wt. %, from about 57 wt. % to about 63 wt. %, from about 58 wt. % to about 62 wt. %, from about 59 wt. % to about 61 wt. %, or about 60 wt. %, based on the total weight of the emulsion (the total weight of the aqueous phase and the oil phase).

Additionally, the aqueous phase can have an acacia gum concentration from about 5 wt. % to about 20 wt. % from about 8 wt. % to about 15 wt. %, from about 10 wt. % to about 14 wt. %, from about 11 wt. % to about 13 wt. %, or about 12 wt. %, based on the total emulsion weight (the total weight of the aqueous phase and the oil phase).

In some cases, the balance between the two are important to consider due to the tackiness, size of the spray dry material and stability of the product. The essential oil needs a good stable tight spray dry material to avoid loss and protect it from volatizing during the spray drying process.

While Gum Acacia Seyal can have less effective emulsification capabilities, it does have advantageous properties for retention of agents, particularly volatile agents such as essential oils. In contrast, Gum Acacia Senegal has highly effective emulsification capabilities while its retention capabilities, especially for volatile agents (e.g., essential oils) is lower. Consequently, a mixture of Gum Acacia Seyal and Gum Acacia Senegal could be advantageous to provide sufficient emulsification capability and sufficient retention of volatile agents in the emulsion.

The oil phase of the emulsion used in the spray drying process can have an oil concentration (e.g., essential oil or other oil described herein) of from about 15 wt. % to about 35 wt. %, from about 17 wt. % to about 33 wt. %, from about 18 wt. % to about 30 wt. %, from about 18 wt. % to about 28 wt. %, from about 18 wt. % to about 25 wt. %, from about 18 wt. % to about 22 wt. %, from about 20 wt. % to about 30 wt. %, from about 20 wt. % to about 28 wt. %, from about 20 wt. % to about 26 wt. %, based on the total weight of the emulsion (the total weight of the aqueous phase and the oil phase).

The oil phase can further comprise soy lecithin at a concentration of from about 0 wt. % to about 4 wt. %, from about 0.5 wt. % to about 3 wt. %, from about 0.5 wt. % to about 2 wt. %, from about 1 wt. % to about 4 wt. %, from about 1 wt. % to about 3 wt. %, from about 1 wt. % to about 2 wt. %, based on the total weight of the emulsion (the total weight of the aqueous phase and the oil phase).

Additionally, the oil phase can further comprise zeolite at a concentration of from about 0 wt. % to about 10 wt. %, from about 1 wt. % to about 8 wt. %, from about 1 wt. % to about 6 wt. %, from about 3 wt. % to about 10 wt. %, from about 3 wt. % to about 8 wt. %, from about 3 wt. % to about 6 wt. %, from about 4 wt. % to about 6 wt. %, or from about 5 wt. % to about 6 wt. %, based on the total weight of the emulsion (the total weight of the aqueous phase and the oil phase).

To prepare the emulsion, the acacia gum is slowly added to the water with moderate mixing and allowed to deaerate for several hours. The phospholipid (e.g., soy lecithin), if desired, is added to the oil and mixed until the mixture is clear and then zeolite is added if desired and mixed until the dispersion is uniform. The ageous phase and the oil phase are added together with homogenization. The homogenization is performed to make an emulsion droplet having a size of from about 800 nm to about 10 microns. The emulsion is then fed into the spray drying process while stirring is maintained to keep a uniform droplet size.

The spray drying process can be any spray drying process that results in a spray dried composition. For example, a conventional spray drying process using the temperatures and pressures of air described below can be used. Additionally, a spray drying process using nitrogen as the propellant can be used and the temperature needed can be slightly lower.

The spray dried particles formed when zeolite particles are not present in the emulsion that is spray dried can have a size in one dimension of from about 200 nm to about 10 microns, from about 200 nm to about 5 microns, from about 200 nm to about 1 micron, from about 200 nm to about 900 nm, from about 200 nm to about 800 nm, from about 300 nm to about 10 microns, from about 300 nm to about 5 microns, from about 300 nm to about 1 micron, from about 300 nm to about 900 nm, from about 300 nm to about 800 nm, from about 400 nm to about 10 microns, from about 400 nm to about 5 microns, from about 400 nm to about 1 micron, from about 400 nm to about 900 nm, from about 400 nm to about 800 nm. The d(50) of the spray dried particles can be from about 500 nm to about 700 nm or about 600 nm.

The heating and shearing can occur at about 20° C. to about 80° C. to keep the compositions below 10,000 Centipoise before spray drying.

The spray drying can occur at about 100° C. to about 1000° C.

The zeolite particles used can be about 5 microns to about 300 microns, about 50 microns to about 200 microns, about 70 microns to about 150 microns, about 80 microns to about 100 microns, or about 10 microns to about 50 microns.

The instant disclosure is further directed to the dry powder compositions made according to these methods.

The resulting dry powder compositions or spray dried compositions can have the following concentrations.

The spray dried compositions can have an acacia gum concentration from about 12.5 wt. % to about 50 wt. % from about 20 wt. % to about 37.5 wt. %, from about 25 wt. % to about 35 wt. %, from about 27.5 wt. % to about 32.5 wt. %, or about 30 wt. %, based on the total spray dried composition weight.

The spray dried compositions can have an oil concentration (e.g., essential oil or other oil described herein) of from about 37.5 wt. % to about 87.5 wt. %, from about 42.5 wt. % to about 82.5 wt. %, from about 45 wt. % to about 75 wt. %, from about 45 wt. % to about 70 wt. %, from about 45 wt. % to about 62.5 wt. %, from about 45 wt. % to about 55 wt. %, from about 50 wt. % to about 75 wt. %, from about 50 wt. % to about 70 wt. %, from about 40 wt. % to about 65 wt. %, based on the total spray dried composition weight.

The spray dried compositions can have soy lecithin (i.e., phospholipid) at a concentration of from about 0 wt. % to about 10 wt. %, from about 1 wt. % to about 7.5 wt. %, from about 1 wt. % to about 5 wt. %, from about 2.5 wt. % to about 10 wt. %, from about 2.5 wt. % to about 7.5 wt. %, from about 2.5 wt. % to about 5 wt. %, based on the total spray dried composition weight.

Additionally, the spray dried compositions can have zeolite at a concentration of from about 0 wt. % to about 25 wt. %, from about 2.5 wt. % to about 20 wt. %, from about 2.5 wt. % to about 15 wt. %, from about 7.5 wt. % to about 25 wt. %, from about 7.5 wt. % to about 20 wt. %, from about 7.5 wt. % to about 15 wt. %, from about 10 wt. % to about 15 wt. %, or from about 12.5 wt. % to about 15 wt. %, based on the total spray dried composition weight.

### Advantages

Without being limited by theory, it is thought that various factors affect the release of the essential oil from the composition. Changes in pH, water presence, and salt presence can cause the release of the essential oil on the skin of the subject from the composition. For example, perspiration results in increased salt and lactic acid concentrations on skin, which could contribute to essential oil release.

When the composition further comprises at least one additional polysaccharide coating, this polymer composition with polysaccharides of different molecular weights can result in a composition with a different porosity, affecting the release rate as well. For example, carrageenan and hydrolyzed starch have lower molecular weights than acacia gum, which results in a more porous composition.

A further advantage of the instant compositions is that they allow for gradual release of the essential oil with a targeted rate of release of about 0.25% to about 20% release rate per hour. This allows for less product waste and fewer reapplications. For example, the instant compositions can have a release rate of up to 90% per hour when emulsified in water.

While diatomaceous earth might be used in similar applications, zeolite is substantially different from diatomaceous earth. One example is in pore size, where zeolite can have pore sizes of 1-10 angstroms, while diatomaceous earth can have pore sizes of 20-40 nm. The two are also formed very differently. Zeolite is formed by gases evacuating from igneous rock when cooling in saltwater, while diatomaceous earth is derived from the fossilized remains of diatoms.

As used in this application, including the appended claims, the singular forms “a,” “an,” and “the” include plural references unless the content clearly dictates otherwise, and are used interchangeably with “at least one” and “one or more.”

The invention will be further described in the following examples, which do not limit the scope of the invention described in the claims.

### EXAMPLES

It is to be understood that while the invention has been described in conjunction with the detailed description thereof, the preceding description is intended to illustrate and not limit the scope of the invention, which is defined by the scope of the appended claims. Other aspects, advantages, and modifications are within the scope of the following claims.

### Example 1. Insect Repellent Compositions

The compositions disclosed above as spray dried compositions, dissolved spray dry compositions, emulsions, and oil formulations can be prepared by mixing the components and in the cases where the spray drying process is used, then those compositions are spray dried according to the process described herein.

### Example 2: Insect Repellency Results

Mosquito Testing to determine repellency. An insect cage was constructed of wood and screen material 8×8×10 inches in size with a clear window in front and covered sides. The back remains open for ventilation for the insect of study, Aegis Aegypti (Yellow Fever Mosquitoes). The control, untreated and samples which were dosed were to be applied to a cut out on top of the cage measuring approximately 0.5 inches smaller then sample membrane, VitroSkin.

Samples were prepared by cutting a 3×5 inch VitroSkin membrane. They were setup in a sealed container with glycerin and water for 18-24 hours to absorb the moisture from the air with a Relative Humidity (RH) of 58-65% RH.

After vortexing the repellent sample, the sample skin was removed from the chamber and then dosed by applying an approximate 1.0-1.5 gram/600 cm². After dosing, the sample was put back into the chamber to maintain consistent humidity throughout the testing. The same goes with the untreated samples (control) by staying in the chamber until testing commences.

Mosquitoes were introduced to the cage and the number was determined based on the amount of landings that occurred within a 5 minute window. The control sample determined this number by observing between 25-50 mosquitoes landings within that time frame before testing the samples. Once the landings were determined and the population feels comfortable for good results for the control, the test was carried out.

The testing started with the control followed by the sample for 5 minutes. The counting of mosquito landings were recorded every hour on the hour until they began to fail repellency at a 89% or less. The repellency was calculated by taking 100 and dividing it by the number of landings for the control and multiplying by the landings. That number is subtracted from 100 to give you the percentage.

Yellow Fever Mosquitoes (Aedes aegypti)
Average Landings and Percent Repellency

| | 30 min | | | 2 hr | | | 4 hr | | |
|---|---|---|---|---|---|---|---|---|---|
| | Pre - | | | Pre - | | | Pre - | | |
| | Treat- | Treat- | % | Treat- | Treat- | % | Treat- | Treat- | % |
| Test | ment | ment | Repel- | ment | ment | Repel- | ment | ment | Repel- |
| Substance: | Landings | Landings | lency | Landings | Landings | lency | Landings | Landings | lency |
| Control- | 35 | 43 | −24% | | | | | | |
| Untreated | | | | | | | | | |
| 15-016 | 41 | 0 | 100% | 39 | 1 | 98% | 43 | 1 | 97% |
| (1.5 g/600 | | | | | | | | | |
| sq. cm.) | | | | | | | | | |
| 15-017 | 32 | 0 | 100% | 38 | 0 | 100% | 39 | 0 | 100% |
| (1.5 g/600 | | | | | | | | | |
| sq. cm.) | | | | | | | | | |
| 15-018 | 40 | 0 | 100% | 40 | 0 | 100% | 40 | 0 | 100% |
| (1.5 g/600 | | | | | | | | | |
| sq. cm.) | | | | | | | | | |
| 15-019 | 48 | 1 | 98% | 42 | 2 | 95% | 35 | 4 | 90% |
| (1.5 g/600 | | | | | | | | | |
| sq. cm.) | | | | | | | | | |
| 15-020 | 47 | 6 | 87% | 44 | 17 | 62% | 47 | 22 | 55% |
| (1.5 g/600 | | | | | | | | | |
| sq. cm.) | | | | | | | | | |
| 15-022 | 42 | 4 | 90% | 35 | 3 | 93% | 46 | 4 | 92% |
| (1.5 g/600 | | | | | | | | | |
| sq. cm.) | | | | | | | | | |

| | 6 hr | | | 8 hr | | |
|---|---|---|---|---|---|---|
| | Pre - | | | Pre - | | |
| | Treat- | Treat- | % | Treat- | Treat- | % |
| Test | ment | ment | Repel- | ment | ment | Repel- |
| Substance: | Landings | Landings | lency | Landings | Landings | lency |
| Control- | | | | | | |
| Untreated | | | | | | |
| 15-016 | 47 | 2 | 96% | 45 | 3 | 93% |
| (1.5 g/600 | | | | | | |
| sq. cm.) | | | | | | |
| 15-017 | 43 | 0 | 100% | 36 | 0 | 100% |
| (1.5 g/600 | | | | | | |
| sq. cm.) | | | | | | |
| 15-018 | 41 | 0 | 100% | 34 | 0 | 100% |
| (1.5 g/600 | | | | | | |
| sq. cm.) | | | | | | |
| 15-019 | 38 | 11 | 70% | 36 | 13 | 66% |
| (1.5 g/600 | | | | | | |
| sq. cm.) | | | | | | |
| 15-020 | 33 | 16 | 53% | 37 | 24 | 38% |
| (1.5 g/600 | | | | | | |
| sq. cm.) | | | | | | |
| 15-022 | 34 | 10 | 70% | 29 | 7 | 77% |
| (1.5 g/600 | | | | | | |
| sq. cm.) | | | | | | |

### Example 3: Process and Preparation of Zeolite to Maximize Desorption, Diffusion, and SPF Value

Zeolite exists in a natural state or can be synthesized. The process starts with a fairly large material between about 5 microns to about 300 microns. In some cases, the zeolite's pore size is between 4.0-7.0 angstroms. Before drying, zeolite particles can contain about 10% moisture by weight.

The process of preparing the zeolite involves drying of the product with less than 70% but preferably at a 50% humidity to draw out the water from the pores.

The zeolite is then added to the specific oils (EO Blend) containing an acid (Lauric Acid) for thickening of the oils in addition to a hydrogenated oil (as a core modifier). This combination needs to be heated to help with drying or dehydration of the zeolite. This decreases the viscosity for easier uptake of the oil, which thickens upon cooling in and on the surface of the zeolite. Using a thickener in up to 35% of the composition adds to the slowing evaporation rate. Adding an additional 5% processing aid can help keep moisture from absorbing over time.

The combination is then put into a shearing process while heating to increase absorption of the oil and thicken the overall composition prior to spray drying. The target particle size is a D50 particle size between 1-10 microns in size.

Spray drying is preferably done while holding heat of the composition at 45-60° C. This is not too hot to allow for desorption but is within the range to continue the dehydration process of the zeolite while maintaining a lower viscosity to help the spray dry process work more efficiently.

For the composition, 10-50 wt. % of the composition material is targeted to be comprised of singularly acacia gum or other polysaccharide as an adjuvant and coating with additional adjuvants and coatings like alginate, carrageenan, alpha-cyclodextrin, starch, and carob. This allows for delayed release as a primary or secondary processing addition to the original core modifier when spray drying.

The powder is then collected and stored.

Powder is added to a dry formulation or left as a powder and added to polymeric spinning or textiles. This maintains the fragrance oils' integrity until water is introduced to the gum or polymer after oil is absorbed onto a substrate or desorbed by evaporation.

Carrier oils include oils and core modifiers of a fatty alcohol, a saturated fatty acid, a medium-chain fatty acid (MCFA), a carboxylic acid, or a combination thereof. The addition of hard oils and waxes as core modifiers in the EO Blend extend release of the essential oil. Longevity and repellency is increased by 10-fold or greater with addition of modifiers like the thickeners used. As the gum or polysaccharide content of the composition increases, the overall longevity of the composition increases significantly.

### Example 4: Sunscreen Coacervate with BEMT Formulation

Table 4A discloses a sunscreen coacervate with BEMT formulation. It is a dry composition that is made by combining the ingredients, heating them to 85° C., and homogenizing while maintaining heat.

TABLE 4A
Sunscreen Coacervate with BEMT Formulation

| Ingredients | INCI | CAS | % | Amount (g) |
|---|---|---|---|---|
| Sunsolv BOS | Butyl | 190085-41- | 10.80 | 21.6 |
| | Octylsalicylate | 7 | | |
| JEESCREEN | Avobenzone | 70356-09-1 | 6.50 | 13.0 |
| A | | | | |
| JEESCREEN | Homosalate | 118-56-9 | 23.00 | 46.0 |
| HMS | | | | |
| Parsol Shield | BEMT | 187393-00- | 14.50 | 29.0 |
| | | 6 | | |
| ESP | Octocrylene | 6197-30-4 | 11.59 | 23.2 |
| Octocrylene | | | | |
| Gslaorb | Octylsalicylate | 118-60-5 | 11.80 | 23.6 |
| Octyl | | | | |
| Salicylate | | | | |
| Z-Cote | Zinc Oxide | 1314-13-2, | 10.95 | 21.9 |
| | | 3397-16-8 | | |
| Carnuba | Carnuba Wax | 8015-86-9 | 10.86 | 21.7 |
| Wax | | | | |
| Aerosil 972 | Silicon | 112945-52- | 0.00 | 0.0 |
| | Dioxide | 5 | | |
| Total | | | 100 | 200 |

### Example 5. Insect Repellent Oil for Spray Dry

Table 5A discloses an insect repellent to be made as a spray dry composition.

TABLE 5A
Insect Repellent Oil for Spray Dry

| Ingredients | % | Amount (g) |
|---|---|---|
| Part A | | |
| Geraniol | 24.70 | 175.4 |
| Vanillin | 4.00 | 28.4 |
| Lauric Acid | 9.30 | 66.0 |
| Castorlatum | 7.40 | 52.5 |
| Part B | | |
| Peppermint Oil | 2.90 | 20.6 |
| Rosemary Oil | 2.56 | 18.2 |
| Citronella Oil | 1.98 | 14.1 |
| Cedarwood Oil | 2.08 | 14.8 |
| Lemongrass Oil | 6.00 | 42.6 |
| Clove Oil | 1.00 | 7.1 |
| Part C | | |
| Zeolite | 38.08 | 270.4 |
| Total | 100 | 710 |

### Example 6: Spray Dry Composition

Composition with Zeolite. An oil phase comprising 70 grams citronella oil, 29 grams zeolite, and 1 gram ALCOLEC® S is prepared by combining the components and mixing the oil phase at high shear to disperse the zeolite.

An aqueous phase is prepared by combining 60 grams water and 30 grams acacia gum and dispersing the acacia gum in the water by mixing at 50° C.

The emulsion is prepared by combining 70 g of the oil phase and 90 grams of the aqueous phase and mix the phases with high shear to product droplets having a diameter of about 1 to about 5 microns.

This emulsion is fed into a spray drier that has an inlet temperature of about 125° C. to about 135° C., a pressure of 3 bars, an outlet temperature of 85° C., and a rate of throughput of 2 kilogram/hour. The resulting spray dried particles had a diameter (or size) of from about 5 microns to about 50 microns, with an average of about 25 microns.

Composition without Zeolite. An oil phase comprising 70 grams citronella oil and 1 gram ALCOLEC® S is prepared by combining the components and mixing the oil phase at high shear to disperse the components.

An aqueous phase is prepared by combining 60 grams water and 30 grams acacia gum and dispersing the acacia gum in the water by mixing at 50° C.

The emulsion is prepared by combining 70 g of the oil phase and 90 grams of the aqueous phase and mix the phases with high shear to product droplets having a diameter of about 1 to about 5 microns.

This emulsion is fed into a spray drier that has an inlet temperature of about 125° C. to about 135° C., a pressure of 3 bars, an outlet temperature of 85° C., and a rate of throughput of 2 kilogram/hour. The resulting spray dried particles had a diameter (or size) of from about 5 microns to about 50 microns, with an average of about 25 microns.

### Example 7: Spray Dry Compositions

| | Formu- | | Formu- | | Formu- | Formu- |
|---|---|---|---|---|---|---|
| Component | la 1 | | la 2 | | la 3 | la 4 |
| Acacia (ENCAPCIA ®)/ | 72 | g | 72 | g | 72 g | 72 g |
| Water | | | | | | |
| Oil | 20.4 | g | 20.4 | g | 28 g | 26 g |
| ALCOLEC-S ® (Soy | 0 | g | 2 | g | 0 g | 2 g |
| Lecithin) | | | | | | |
| Zeolite | 5.6 | g | 5.6 | g | 0 g | 0 g |

The ENCAPCIA® acacia gum is added to water with moderate mixing and allowed to deaerate for several hours. The phospholipid (i.e. soy lecithin, ALCOLEC-S®) is added at the desired concentration to the oil and mixed until the mixture is clear. The zeolite is added to the phospholipid and oil phase with mixing until the dispersion is uniform. The oil phase and the water phases are combined with homogenization to prepare an emulsion droplet having a size in one dimension of about 800 nm to about 10 microns. The emulsion is used in a spray dry process, for example, these emulsions can be used in the spray dry process described in Example 6.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compounds, compositions, and methods without departing from the scope of the invention, it is intended that all matter contained in the above description and shown in the accompanying drawings shall be interpreted as illustrative and not in a limiting sense.

1. A composition comprising:
one or more polymers;
from about 20 wt. % to about 80 wt. % of one or more medium chain fatty acids or salts or esters thereof having 6-12 carbon atoms;
one or more active agents;
wherein the active agent and the medium chain fatty acid or salt or ester thereof are coated or partially coated with the polymer.

2. (canceled)

3. The composition of claim 1, wherein the polymer comprises acacia gum, corn starch, carrageenan, hyaluronic acid, pectin, alginate, chitosan, guar gum, arabic gum, xanthan gum, carboxymethylcellulose, carob bean gum, cellulose, alpha-cyclodextrin, polylactic acid, a vinyl polymer, polyethylteraphthalate, or a combination thereof.

4.-8. (canceled)

9. The composition of claim 1, wherein the medium chain fatty acid or salt or ester thereof is present in palm kernel oil, coconut oil, or a combination thereof.

10.-17. (canceled)

18. The composition of claim 1, wherein the active agent comprises an antibacterial, a color compound, a fragrance compound, an active pharmaceutical ingredient (API), an over-the-counter (OTC) ingredient, an antioxidant, a flavinoid, an essential oil, or a combination thereof.

19.-21. (canceled)

22. The composition of claim 1, wherein the active agent comprises an essential oil.

23.-37. (canceled)

38. A dry spray dried powder composition comprising:
a) molecular sieves impregnated with at least one essential oil; and
b) at least one free essential oil;
wherein the at least one free essential oil and molecular sieves are coated or partially coated with at least one polysaccharide.

39. The composition of claim 38, wherein the molecular sieve comprises one or more zeolite particles.

40.-44. (canceled)

45. The composition of claim 39, wherein the zeolite particles are derived from about 71.7% Si to about 91.7% Si, from about 5% Al to about 15% Al, from about 4% K to about 10% K, from about 0.5% Fe to about 1.5% Fe, from about 0.5% Ca to about 1.5% Ca, from about 0.25% Mg to about 1.25% Mg, and from about 0.75% Na to about 1.75% Na.

46. (canceled)

47. A dry topical composition comprising the composition of claim 1 and at least one carrier.

48.-60. (canceled)

61. The topical composition of claim 47, wherein the topical composition is sunscreen, insect repellent, or combination sunscreen insect repellent.

62. (canceled)

63. The topical composition of claim 61, wherein the topical composition is a combination sunscreen and insect repellent.

64.-67. (canceled)

68. A method of repelling insects from a subject, the method comprising administering the insect repellent composition of claim 61 to the subject's skin or near a subject.

69. (canceled)

70. A dry sunscreen composition comprising the composition of claim 47 and a sun-blocking agent.

71.-73. (canceled)

74. A method of blocking sun exposure of a subject, the method comprising administering the sunscreen composition of claim 70 to the subject.

75.-78. (canceled)

79. A spray-dried composition derived from spray drying an emulsion comprising an oil phase and an aqueous phase, wherein
the aqueous phase comprises from about 50 wt. % to about 70 wt. % water and from about 5 wt. % to about 20 wt. % acacia gum, based on the total weight of the emulsion; and
the oil phase comprises from about 15 wt. % to about 35 wt. % oil, based on the total weight of the emulsion.

80. The spray-dried composition of claim 79, wherein the oil phase further comprises from about 0.5 wt. % to about 4 wt. % phospholipid, based on the total weight of the emulsion.

81. The spray-dried composition of claim 80, wherein the oil phase further comprises from about 1 wt. % to about 10 wt. % zeolite, based on the total weight of the emulsion.

82. (canceled)

83. The spray-dried composition of claim 80, wherein the aqueous phase comprises from about 10 wt. % to about 14 wt. % acacia gum, based on the total weight of the emulsion.

84.-88. (canceled)

89. A spray-dried composition comprising
from about 12.5 wt. % to about 50 wt. % acacia gum;
from about 37.5 wt. % to about 70 wt. % oil; and
from about 0.1 wt. % to about 5 wt. % phospholipid,
based on the total weight of the spray-dried composition.

90.-95. (canceled)

96. A method of producing a dry powder composition, the method comprising:
a) drying zeolite particles at 70% or less humidity;
b) combining the zeolite particles with an essential oil composition comprising a thickener, at least one essential oil, and a hydrogenated oil to generate an essential oil-zeolite particles composition;
c) heating and shearing the essential oil-zeolite particle composition; and
d) combining the essential oil-coated zeolite particle composition with a polysaccharide composition comprising at least one polysaccharide and spray drying the combination to generate the dry powder composition.

97.-102. (canceled)
